(19) United States
(12) Patent Application Publication
(10) Pub. No.: US 2011/0189248 A1
(43) Pub. Date: Aug. 4, 2011

(54) Home And Personal Care Compositions

(76) Inventors: Eva Baldaro, Milano (IT); Sylvie Bouzeloc, Montigny-E-Tilleul (BE); Stephanie Postiaux, Epinois (BE); Esterina Schirosi, Anderlues (BE); Dirk Soontjens, Mumbai (IN); Stephane Ugazio, Soignies (BE)

(21) Appl. No.: 12/994,486

(22) PCT Filed: Jul. 14, 2009
(86) PCT No.: PCT/US2009/050453

§ 371 Date: Mar. 25, 2011

(30) Foreign Application Priority Data
Jul. 18, 2008 (GB) 0813140.1

(51) Int. Cl. A61K 8/89; A61K 8/898; A61Q 5/02; A61Q 19/10; C11D 7/60; A61Q 5/12; A61K 8/02; A61Q 19/00; A61Q 5/00; A61K 8/92

(52) U.S. Cl. 424/401; 424/70.12; 424/70.122; 514/54; 424/78.03; 510/122; 510/158; 510/515

(57) ABSTRACT

Provided are home and personal care compositions comprising a cationic hydrophobically modified galactomannan ether and a silicone, where the cationic hydrophobically modified galactomannan ether have a cationic degree of substitution (DS_{cat}) from 0.01 to 0.5 and a hydrophobic degree of substitution (DS_{H}) from 0.00001 to less than 0.001 These compositions provide a benefit to hair, skin or fabric onto which they have been applied. Benefits may include softness, level of silicone deposition and ease of detangling.

The present invention relates to home and personal care compositions, particularly those containing a galactomannan ether, especially a cationic hydrophobically modified galactomannan ether and a silicone. Such compositions tend to provide desirable characteristics.

Galactomannan ethers such as guars have been known to be used in home and personal care compositions in combination with silicone.

Home and personal care compositions such as conditioning shampoos, shower gel and fabric care compositions containing conditioning agents of various types have been disclosed before and are well known by the man skilled in the art to allow for the cleaning and conditioning of hair, skin and fabric.

U.S. Pat. No. 5,085,857 describes an aqueous shampoo composition comprising among essential ingredients a cationic conditioning polymer which is guar hydroxypropyltrimonium chloride and an insoluble, non-volatile silicone, present as emulsified particles with an average particle size of less than 2 μm.

U.S. Pat. No. 6,355,234 describes an aqueous shampoo composition comprising, in addition to water a surfactant, emulsified particles of an insoluble, non-volatile silicone selected from the group consisting of polyalkyl siloxanes, polyalkyl aryl siloxanes and silicone gums, and a soluble cationic hair conditioning polymer having a cationic charge density of about +3.0 meq/g or less, in which the emulsified particles of insoluble, non-volatile silicone are incorporated into the shampoo composition as a preformed aqueous emulsion having an average silicone particle size in the emulsion and in the shampoo composition of from 2 to 30 microns.

U.S. Pat. No. 6,387,855 describes a composition for washing and conditioning keratin substances comprising, in a cosmetically acceptable aqueous medium, at least one silicone, at least one surfactant with detergent properties, and at least one hydrophobic galactomannan gum, wherein said at least one hydrophobic galactomannan gum comprises hydrophobic substituents chosen from linear or branched alkyl groups containing from 8 to 60 carbon atoms, linear or branched alkenyl groups containing from 8 to 60 carbon atoms, and mixtures thereof, wherein said alkyl and alkenyl groups can be substituted with one or more hydroxyl groups.

U.S. Pat. No. 6,930,078 describes a shampoo composition comprising from about 5 to about 50 weight percent of a detersive surfactant, at least about 0.05 weight percent of a cationic guar derivative; wherein said cationic guar derivative has a molecular weight from about 10,000 to about 10,000,000; and wherein said cationic guar derivative has a charge density from about 1.5 meq/g to about 7 meq/g; at least about 0.1 weight percent of particles having a mean particle size of less than about 300 microns, wherein said particles are selected from the group consisting of hollow particles, solid particles and combinations thereof; and at least about 20.0 weight percent of an aqueous carrier. The composition further comprises a conditioning agent selected from the group consisting of silicone oils, cationic silicones, silicone gums, high refractive index silicones, silicone resins, hydrocarbon oils, polyolefins, fatty esters and mixtures thereof.

EP 1513485 describes a shampoo composition comprising among essential ingredients, a cationic polymer, such as a cationic guar derivative, wherein said cationic polymer has a molecular weight from about 10,000 to about 10,000,000 and a charge density from about 1.4 meq/g to about 7 meq/g, and a conditioning agent material having a volume average diameter of less than about 1.0 microns, such as an organosiloxane nanoemulsion.

U.S. Pat. No. 5,135,748 describes an aqueous composition comprising, among essential ingredients, a cationic polysaccharide, which is substituted with a quaternary ammonium group having at least one substituent on the nitrogen being an alkyl group from 12 to 22 carbon atoms in length. Particularly preferred in the specification are polymeric quaternary ammonium salts of hydroxyethyl cellulose reacted with a fatty alkyl dimethyl ammonium substituted epoxide. For improved lubricity, there may also be included one or more silicone oils or fluids which may be selected from a dimethyl polysiloxane, including a polysiloxane end-blocked with trimethyl units and polydimethylcyclosiloxane.

EP 1690524 describes cleaning and conditioning means for skin and hair, comprising, among other ingredients, a quaternized hydroxyethylcellulose polymer, hydrophobically modified with dimethyldodecylammonium groups and non-ionic or cationic polymer, differing from the previous. The composition may also contain an insoluble silicone fluid.

US 2007/0258918 describes a hair conditioner composition consisting essentially of a cationic polymer with a mean average molecular weight from about 2,000 to about 10,000,000 Dalton, and a cationic degree of substitution greater than 0.25 to about 3.0, and at least one hair conditioner active ingredient, wherein the hair conditioner composition is substantially surfactant-free.

EP 1558718 relates to a liquid laundry detergent composition comprising among other essential ingredients at least one detergent ingredient selected from the group consisting of anionic surfactant, zwitterionic surfactant, amphoteric surfactant and mixtures thereof; a coacervate phase forming cationic polymer such as cationic guar gums and derivatives thereof, more preferably chloride salts of cationic guar hydroxypropyltriammonium; and one or more cationic silicone polymers.

US 2006/0030513 is directed to laundry compositions which deliver both effective softening and effective cleaning, containing: (a) a cationic polymer having a weight average molecular weight of less than about 850,000 daltons, such as guar hydroxypropyltrimonium chloride; (b) about 1% to about 60% of a nonionic oil, such as a silicone oil; and (c) at least about 5% of a surfactant selected from the group consisting of anionic surfactant, cationic monomeric surfactant, nonionic surfactant, zwitterionic surfactant, and combinations thereof; wherein the ratio of said cationic polymer to said nonionic oil is less than about 0.25; wherein the ratio of cationic monomeric surfactant to said nonionic oil is less than about 0.2; and having a Softening Parameter of greater than about 70.

U.S. Pat. No. 5,500,152 describes shower gel compositions in the form of a liquid or gel comprising 5-50% wt. of a detergent comprising 30-100% wt. (on total detergent) of a fatty acyl isethionate and 0-70% wt (on total detergent) of other detergent, 0.01-5% on product of cationic polymer and 0.5-15% wt. by weight of silicone. The compositions show enhanced silicone deposition as compared with composition employing other surfactants. An example of a suitable cationic polymer is the hydroxypropyltrimethylammonium derivative of guar gum (CTFA designation Guar Hydroxypropyltrimonium Chloride). Suitable silicones include polyalkyl or polyaryl siloxanes.

US 2007/0148116 relates to a concentrated ingredient for treating and/or modifying the skin and/or the hair. The invention also relates to the use of this ingredient in cosmetic compositions, for example in shampoos, shower gels or leave-in or rinse-out hair conditioners. The ingredient comprises a conditioning agent and a polymer for aiding deposition. The conditioning agent may be chosen especially from: a1) plant, mineral or animal oils, or derivatives thereof, and a2) polyorganosiloxanes. The polymer for aiding deposition may be chosen especially from: b1) derivatives of natural polymers comprising cationic or potentially cationic groups, for example cationic cellulose, guar or starch derivatives, and b2) synthetic polymers comprising cationic or potentially cationic groups, and zwitterionic groups.

There is a continuing need for further improvement and it has now been surprisingly found that compositions containing certain cationic hydrophobically modified galactomannan ethers in combination with silicone may provide one or more of the following benefits: improved conditioning properties, improved softness and/or increased silicone deposition.

Galactomannans are polysaccharides composed mainly of galactose and mannose units. The main source of galactomannans is the endosperm of certain leguminous seeds such as guar, carob, tara, cassia obtusifolia and the like. In particular, the polysaccharide contained in guar seeds consists of a main chain of mannose units linked together by 1-4-β-glycosidic linkages from which single galactose units branch by means of 1-6-α-glycosidic linkages. The ratio of galactose units to mannose units can vary from one source to another. In the case of the polysaccharide contained in guar seeds the ratio is about 1:2.

Cellulose constitutes another class of polysaccharides, composed of repeated glucose units. The chemical structure difference, compared to the galactomannans of the present invention, ultimately impact the chemical reactivity of the initial polymers and viscosity behaviour of the modified polymers.

It has now been found that cationic hydrophobically modified galactomannan ethers with certain degrees of cationic and hydrophobic substitution, preferably obtained via a two step reaction using a hydrophobing etherifying agent and a cationizing etherifying agent are especially effective as stabilizers and conditioning agents for use in home and personal care compositions, especially cleaning products.

Accordingly the invention provides home and personal care compositions comprising a silicone and a cationic hydrophobically modified galactomannan ether have a cationic degree of substitution (DS_{cat}) from 0.01 to 0.5 and a hydrophobic degree of substitution (DS_{H}) from 0.00001 to less than 0.001. Such galactomannan ethers are themselves novel materials and are the subject of a separate patent application.

In the present text, with the expression “cationic degree of substitution” (DS_{cat}) we mean the cationic substitution on the hydroxyl groups of galactomannans, particularly guar, measured by means of ¹H-NMR and with the expression “hydrophobic degree of substitution” (DS_{H}) we mean the hydrophobic substitution on the hydroxyl groups of galactomannans, especially guar, measured by means of gas chromatography; the hydroxyethyl and/or hydroxypropyl molar substitution degree is also measured by means of ¹H-NMR. In the most preferred compositions according to the invention the cationic hydrophobically modified galactomannan ethers used have DS_{cat} from 0.1 to 0.3 and DS_{H} between 0.00005 and 0.0009. Particularly useful are compositions where the hydrophobically modified cationic galactomannan ethers have a DS_{cat} from 0.1 to 0.3, a DS_{H} between 0.00005 and 0.0009, and hydroxypropyl molar substitution from 0.3 to 1.2.

The cationic substituents of the galactomannan ethers derive from the reaction of one of the hydroxyl group of the galactomannan with tertiary amino or quaternary ammonium alkylating agents, such as 2-dialkylaminoethyl chloride and quaternary ammonium compounds such as 3-chloro-2-hydroxypropyltrimethylammonium chloride, and 2,3-epoxy-propyltrimethylammonium chloride. Preferred examples include glycidyltrialkylammonium salts and 3-halo-2-hydroxypropyltrialkylammonium salts such as glycidyltrimethylammonium chloride, glycidyltriethylammonium chloride, gylcidyltripropylammonium chloride, glycidylethyldimethylammonium chloride, glycidyldiethylmethylammonium chloride, and their corresponding iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding iodides; and quaternary ammonium compounds such as halides of imidazoline ring containing compounds.

In particularly suitable compositions, the quaternary ammonium alkylating agent is (3-chloro-2-hydroxypropyl)trimethylammonium chloride. The cationic substituent is in this case 2-hydroxy-3-[trimethylammonium]propyl chloride. These tertiary amino or quaternary ammonium alkylating agents do not normally contain linear or branched alkyl chains having 4 or more carbon atoms.

The hydrophobic modification of the galactomannan ether used in the compositions according to the invention is obtained by the introduction of hydrophobic substituents. The useful hydrophobic substituents do not contain amine groups or quaternary ammonium groups and are chosen from the group consisting of linear or branched alkyl and/or alkenyl substituents of from 12 to 32 carbon atoms either alone or mixed together.

In the preferred galactomannan ethers for use in the compositions according to the invention the cationic substituent is 2-hydroxy-3-(trimethylammonium)propyl chloride and the hydrophobic substituent is a linear chain alkyl containing between 14 and 24 carbon atoms or a mixture of such alkyls. Suitable cationic hydrophobically modified galactomannan ether for use in the compositions according to the invention may further contain hydroxyethyl and/or 2-hydroxypropyl substituent groups, their total molar substitution degree ranging from 0 to 3, although galactomannan containing only the cationic and the hydrophobic substituent are preferred.

The process for preparing the hydrophobically modified cationic galactomannan ethers which are useful in the present invention comprises the hydrophobic modification of a commercially available galactomannan gum, or hydroxyethyl and/or 2-hydroxypropyl galactomannan ether having a molar substitution from 0.3 to 3.0, and the reaction with tertiary amino or quaternary ammonium alkylating agents, preferably with 2,3-epoxypropyl trimethylammonium chloride or (3-chloro-2-hydroxypropyl)trimethylammonium chloride, in the presence of basic catalysts (such as sodium hydroxide). When the cationic hydrophobically modified galactomannan ether also contains hydroxyethyl and/or 2-hydroxypropyl substituents, the hydroxyethyl and/or hydroxypropyl substituents may also be introduced in the last step, after hydrophobing and cationisation of galactomannan, especially guar, gum have occurred.

Therefore an alternative process for preparing hydrophobically modified cationic galactomannan ethers for use in the compositions according to the present invention comprises the hydrophobic modification of a commercially available galactomannan gum, the reaction with tertiary amino or quaternary ammonium alkylating agents, preferably with 2,3-epoxypropyl trimethylammonium chloride or (3-chloro-2-hydroxypropyl)trimethylammonium chloride, and the reaction of the hydrophobically modified cationic intermediate with ethylene oxide and/or propylene oxide, in the presence of basic catalysts (such as sodium hydroxide), to give the cationic hydrophobically modified galactomannan ether containing hydroxyethyl and/or 2-hydroxypropyl substituents.

It has been found that the order in which the cationic and hydrophobic substituents are introduced on the guar backbone is relevant for obtaining the final product. It was particularly found that it was unexpectedly unfavourable to add hydrophobic substituents on cationic galactomannan ethers. Therefore, preferably, the reaction steps take place in the order given above, where the cationisation follows the hydrophobic modification.

The starting polysaccharides for obtaining the hydrophobically modified cationic modified galactomannan ether for use in the compositions according to the invention preferably have a molecular weight typically of from 50,000 to 5,000,000 depending on the polysaccharide origin; preferably the polysaccharide is guar galactomannan. Also, the cationic hydrophobically modified galactomannan ether for use in the compositions according to the invention has a preferred molecular weight of from 50,000 to 5,000,000.

The reactants suitable for the hydrophobic modification include epoxy-alkanes and/or alkenes, alkyl- and/or alkenyl-glycidylethers, alkyl- and/or alkenyl-β-hydroxy-γ-chloropropyl ethers, and epoxy derivatives of triglycerides. It is apparent that with the exception of the case in which the reactant is an alkyl and/or alkenyl halide, the hydrophobic substituent is not a simple alkyl and/or alkenyl radical. In effect, the substituent is a hydroxy-alkyl and/or alkenyl in the case of epoxy-alkanes and/or alkenes; a hydroxy-(oxa)-alkyl and/or alkenyl in the case of glycidyl ethers and β-hydroxy-γ-chloropropyl ethers. Notwithstanding this, the use of the term “alkyl and/or alkenyl substituents” was preferred in that, regarding the properties of the compounds of the present invention, substantial differences have not been noted between one compound and another, as far as they are chosen in the above list.

In a preferred process the cationic hydrophobically modified galactomannan ether is obtained using guar gum, possibly dispersing it in an inert diluent, such as lower aliphatic alcohols, ketones, or liquid hydrocarbons, treating it at ambient temperature with an alkaline hydroxide in aqueous solution and then reacting with one of the said hydrophobing reactants at a temperature of between 40° C. and 80° C. for 1 to 6 hours. On termination of the reaction the system is preferably set to 40° C. and the cationising agent is introduced into the reactor, possibly dispersed in an inert organic diluent, and the reaction is completed by raising the temperature to 50-80° C. for 1 to 3 hours. Where the cationic hydrophobically modified galactomannan ether further contains hydroxyethyl and/or 2-hydroxypropyl substituents a third reaction step would be required (hydroxyalkylating step).

For use in home and personal care compositions according to the invention, it is sometimes preferred that the galactomannan ethers are further subjected to a purification step to obtain a particularly pure product. Such purification step may take place by extraction of the impurities with an organic or aqueous-organic solvent before a final drying step for example in order to remove the salts and by-products formed during the reaction.

Or, alternatively, but preferably, the purification step takes place by glyoxalation after termination of the synthetic steps (cationisation, hydrophobing and, where needed, hydroxyalkylation), as described for example in WO 2008/058768 or WO 2003/078474. The purified product obtained by means of glyoxalation is insoluble at pH lower than 7 and quickly and completely soluble at pH higher than 8; therefore it can be dispersed and dissolved readily in water.

Yet another alternative purification step may also be used, which is done by boron crosslinking of the starting galactomannan gum with sodium tetraborate decahydrate; at the end of the synthetic steps, the product having a pH higher than 9 is then washed with water. The obtained purified product obtained can be dispersed in water, because of its insolubility at pH higher than 9, and quickly and completely dissolved at pH lower than 7.

The particularly useful cationic hydrophobically modified galactomannan ethers having a cationic molar substitution from 0.01 to 0.5, a hydrophobic molar substitution from 0.00001 and below 0.001 are those in which the hydrophobic substituents do not contain amine groups or quaternary ammonium groups and are chosen from the group consisting of linear or branched alkyl and/or alkenyl substituents of between 12 and 32 carbon atoms.

The silicones which may be used in home and personal care compositions according to the invention may be in the form of oils, waxes, resins, elastomers or gums, possibly modified with organic moieties, and may be soluble or non soluble in the home and personal care compositions according to the invention. Of course any combination or mixture of different silicones may also be used. The silicones, e.g. organopolysiloxanes are defined in greater detail in Walter Noll's “Chemistry and Technology of Silicones” (1968) Academic Press. They may be volatile or non-volatile. Such silicones are known to the person skilled in the art as are methods for making them and many of them commercially available.

If volatile, the silicones are more particularly chosen from those having a boiling point below 250° C., and even more particularly from: (i) cyclic silicones containing from 3 to 7 and preferably from 5 to 6 silicon atoms; (ii) linear volatile silicones having 2 to 9 silicon atoms and having a viscosity of less than or equal to 5 mm²/s at 25° C. The volatile silicones may also be mixtures of (i) and (ii).

The volatile silicones may be volatile methyl siloxane or volatile ethyl siloxanes.

Non-volatile silicones, and more particularly polyalkylsiloxanes, polyarylsiloxanes, polyalkylarylsiloxanes, silicone gums and resins, polyorganosiloxanes modified with organofunctional groups, and mixtures thereof, are more preferably used. Such silicones are more particularly chosen from polyalkylsiloxanes, especially polydimethylsiloxanes containing trimethylsilyl end groups (CTFA designation Dimethicone) having a viscosity of from 5 mm²/s to 2.5 million mm²/s at 25° C., and preferably 10 to 1 million mm²/s. Also suitable are polydimethylsiloxanes containing dimethylsilanol end groups (CTFA designation Dimethiconol).

Polyalkylarylsiloxanes which are useful in compositions according to the invention may be chosen, for example, from linear and branched polydimethylmethylphenylsiloxanes and polydimethyldiphenylsiloxanes with a viscosity of from 10 to 50 000 mm²/s at 25° C.

Silicone gums which may be used in compositions according to the invention may be polydiorganosiloxanes having high number-average molecular masses of between 200,000 and 1,000,000, used alone or in conjunction with a solvent. This solvent may be chosen from volatile silicones, polydimethylsiloxane (PDMS) oils, isoparaffins, hydrocarbon solvents, or mixtures thereof. Products which may be used more particularly in accordance with the invention are mixtures such as: mixtures formed from a polydimethylsiloxane hydroxylated at the end of the chain (CTFA designation Dimethiconol) and from a cyclic polydimethylsiloxane (CTFA designation Cyclomethicone).

Organopolysiloxane resins which may be used in accordance with the invention are crosslinked siloxane systems and consist of siloxane units of the general formula R″ₕSiO_{4-h/2} wherein R″ denotes a hydrocarbon-based group having from 1 to 16 carbon atoms or a phenyl group and wherein h may have a value of from 0 to 3, but preferably has an average value of from 0.5 to 2. Crosslinking is obtained by incorporating trifunctional and/or tetrafunctional silanes with the monofunctional silane and/or difunctional silane monomers used during manufacture. The degree of crosslinking required to obtain a suitable silicone resin will vary according to the specifics of the silane monomer units incorporated during manufacture of the silicone resin. Among these products, those particularly preferred are the ones in which R″ denotes a C₁-C₄ lower alkyl radical, more particularly methyl, or a phenyl radical. The organopolysiloxane resins which may be used in accordance with the invention may be used alone or in conjunction with a solvent. Such solvent may be chosen from volatile silicones, polydimethylsiloxane (PDMS) oils, isoparaffins, hydrocarbon solvents, or mixtures thereof. Silicones which may be used more particularly in accordance with the invention are mixtures of multiple types of silicones, such as: mixtures formed from an organopolysiloxane resin (CTFA designation Trimethylsiloxysilicate) and a cyclic or linear polydimethylsiloxane (CTFA designation Cyclomethicone or Dimethicone) or phenyltrimethylsiloxysilane.

Organopolysiloxane resins which may be used in accordance with the invention are those described in U.S. Pat. No. 5,152,984 and U.S. Pat. No. 5,126,126, such as Aminopropyl Phenyl Trimethicone (CTFA designation).

Organomodified silicones which may be used in accordance with the invention are silicones containing in their structure one or more organofunctional groups attached via a Si—C or Si—O—C linkage. Among suitable organomodified silicones are polyorganosiloxanes containing: polyethylenoxy and/or polypropylenoxy groups optionally containing C₆-C₂₄ alkyl groups, such as the products known as PEG/PPG-Dimethicone and the (C₁₂)alkylmethicone copolyol; substituted or unsubstituted amine groups. Substituted amine groups include, in particular, C₁-C₄ aminoalkyl groups.

Organomodified silicones which may be used in accordance with the invention include the aminofunctional polyorganosiloxane having its formula selected from the group consisting of

R²R₂SiO(R₂SiO)ₐ(R¹RSiO)_{b}SiR₂R² and

R²R₂SiO(R₂SiO)ₐ(R¹SiO_{3/2})_{b}SiR₂R²

wherein R is a monovalent hydrocarbon radical, R¹ is an aminoalkyl group having its formula selected from the group consisting of —R³NH₂ and —R³NHR⁴NH₂ wherein R³ is a divalent hydrocarbon radical having at least 3 carbon atoms and R⁴ is a divalent hydrocarbon radical having at least 2 carbon atoms, R² is selected from the group consisting of R, R¹, and —OH, a has a value of 0 to 2000, and b has a value of from greater than zero to 200.

The monovalent R radicals are exemplified by alkyl radicals such as the methyl, ethyl, propyl, butyl, amyl, and hexyl, alkenyl radicals such as the vinyl, allyl, and hexenyl, cycloalkyl radicals such as the cyclobutyl and cyclohexyl, aryl radicals such as the phenyl and naphthyl, aralkyl radicals such as the benzyl and 2-phenylethyl, alkaryl radicals such as the tolyl, and xylyl, halohydrocarbon radicals such as 3-chloropropyl, 4-bromobutyl, 3,3,3-trifluoropropyl, chlorocyclohexyl, bromophenyl, and chlorophenyl. It is preferred that R is a monovalent hydrocarbon radical having from 1 to 6 carbon atoms. Especially preferred R radicals are methyl, phenyl, and vinyl.

The group R³ is preferably an alkylene radical having from 3 to 20 carbon atoms. Preferably R³ is selected from the group consisting of propylene, —CH₂ CHCH₃—, butylene, —CH₂CH(CH₃)CH₂—, pentamethylene, hexamethylene, 3-ethyl-hexamethylene, octamethylene, and decamethylene.

The group R⁴ is preferably an alkylene radical having from 2 to 20 carbon atoms. Preferably R⁴ is selected from the group consisting of ethylene, propylene, —CH₂ CHCH₃—, butylene, —CH₂CH(CH₃)CH₂—, pentamethylene, hexamethylene, 3-ethyl-hexamethylene, octamethylene, and decamethylene.

It is highly preferred in this invention that R¹ is selected from the group consisting of —CH₂CH₂CH₂NHCH₂CH₂NH₂ and —CH₂CH(CH₃)CH₂NHCH₂CH₂NH₂.

Salts of these same aminofunctional radicals may also be used in this invention. Examples of such salts include alkyl carboxylate salts, aryl carboxylate salts, halide salts such as chlorides and bromides, and other neutralization products of the amines with organic acids.

Although the group R² may be selected from the group consisting of R, R¹, and —OH, it is preferred for purposes of this invention that R² is methyl or —OH.

It is preferred that the polyorganosiloxanes have from about 0.1 to 15 molar percent of the above described amino groups and most preferably from about 0.2 to 10 molar percent of the above described amino groups. In the above formulas, preferably a has a value of from 50 to 2000, and b has a value of 1 to 100. The aminofunctional polyorganosiloxanes useful in the this invention may be prepared by procedures well known in the art. Many of these polyorganosiloxanes are available commercially. Therefore their preparation will not be described here.

Other suitable organomodified silicones include those having alkoxylated groups; hydroxyl groups such as the polyorganosiloxanes containing a hydroxyalkyl function, as described in EP 1081272, U.S. Pat. No. 6,171,515 and U.S. Pat. No. 6,136,215.

Organomodified silicones which may be used in accordance with the invention include Bis-Hydroxy/Methoxy Amodimethicone.

Other organomodified silicones which may be used in accordance with the invention are amino-acid functional siloxanes obtained by reacting an amino acid derivative selected from the group of an N-acyl amino acid and an N-aroyl amino acid with an amino functional siloxane, further described in WO 2007/141565.

Other organomodified silicones which may be used in accordance with the invention are quaternary ammonium functional silicones, described in U.S. Pat. No. 6,482,969 and U.S. Pat. No. 6,607,717, such as Silicone Quaternium-16 (CTFA designation).

Some organomodified silicones which may be used in accordance with the invention are water soluble or water dispersible silicone polyether compositions. These are also known as polyalkylene oxide silicone copolymers, silicone poly(oxyalkylene) copolymers, silicone glycol copolymers, or silicone surfactants. These may be linear rake or graft type materials, or ABA and ABn types where the B is the siloxane polymer block, and the A is the poly(oxyalkylene) group. The poly(oxyalkylene) group may consist of polyethylene oxide, polypropylene oxide, or mixed polyethylene oxide/polypropylene oxide groups. Other oxides, such as butylene oxide or phenylene oxide are also possible.

Other organomodified silicones which may be used in accordance with the invention are amino ABn silicone polyether block copolymer, where an amino functionality is added to the ABn silicone polyether copolymer, also described in IP.COM 00141525 such as Bis-Isobutyl PEG/PPG-20/35/Amodimethicone Copolymer (CTFA designation).

Preferred organomodified silicones which may be used in accordance with the invention are those designated by the CTFA dictionary as PEG/PPG-Dimethicone or amodimethicone and also aminopolyether substituted silicone.

Other organomodified silicones which may be used in accordance with the invention are hydrocarbyl functional organopolysiloxanes comprising a siloxy unit of the formula R⁵R′ᵢSiO_{(3-i)/2} wherein R′ is any monovalent hydrocarbon group, but typically is an alkyl, cycloalkyl, alkenyl, alkaryl, aralkyl, or aryl group containing 1-20 carbon atoms, R⁵ is a hydrocarbyl group having the formula —R⁶OCH₂CH₂OH, wherein R⁶ is a divalent hydrocarbon group containing 2 to 6 carbon atoms and i has a value of from zero to 2. Such hydrocarbyl functional organopolysiloxanes are further described in U.S. Pat. No. 2,823,218, U.S. Pat. No. 5,486,566, U.S. Pat. No. 6,060,044 and U.S. Pat. No. 20,020,524. Preferred hydrocarbyl functional organopolysiloxanes which may be used in compositions according to the invention are those designated by CTFA as Bis-Hydroxyethoxypropyl Dimethicone.

Yet another organomodified silicone which may be used in accordance with the invention may be siloxane-based polyamide. U.S. Pat. No. 6,051,216 discloses siloxane-based polyamides as gelling agents for cosmetic products, methods for making such agents, and formulations thereof. Such polyamides contain siloxane groups in the main chain and act to thicken compositions containing volatile and/or non-volatile silicone fluids. Variants of siloxane-based polyamides such as silicone polyether-amide block copolymers described in US 2008/0045687, may also be used in accordance with the invention.

Yet more organomodified silicones which may be used in accordance with the invention may be vinyl-type polymer having a carbosiloxane dendrimer structure on their side molecular chain. These may be used as neat polymer or as a solution or a dispersion in a liquid such as a silicone oil, organic oil, alcohol, or water. Such polymers which may be used in accordance with the invention are further described in EP 0963751, and are given the CTFA designation Acrylates/Polytrimethylsiloxymethacrylate Copolymer.

Other organomodified silicones which may be used in accordance with the invention may be alkylmethylsiloxane materials which may be present as liquids or waxes. In liquid form they can be either cyclic having a structure comprising:

[MeR⁷SiO]ₚ[Me₂SiO]_{q}

or linear having a structure comprising

R⁸Me₂SiO(MeR⁷SiO)_{w}(Me₂SiO)ₓSiR⁸Me₂

wherein each R⁷ is independently a hydrocarbon of 6 to 30 carbon atoms, R⁸ is methyl or

R⁷, p is 1-6, q is 0-5, w is 0-5 and x is 0-5, provided p+q is 3-6 and q is not 0 if R⁸ is methyl. These liquids may be either volatile or non-volatile and they can have a wide range of viscosities such as from about 0.65 to about 50,000 mm²/s at 25° C. Alkylmethylsiloxane waxes have the structure:

R⁸Me₂SiO(Me₂SiO)_{y}(MeR⁷SiO)_{z}SiMe₂R⁸

wherein y is 0-100, z is 1-100, R⁷ is an alkyl group of 6-30 carbon atoms and R⁸ is methyl or R⁷. Preferably, the alkylmethylsiloxane has the formula:

Me₃SiO(Me₂SiO)_{y}(Me R⁷SiO)_{z}SiMe₃

The above alkylmethylsiloxane materials are known in the art and can be produced by known methods.

Silicone elastomers which may be used in compositions according to the invention are crosslinked siloxane systems. Most of these elastomers can be used to cause volatile silicones fluids or low polarity organic solvents such as isododecane to gel. Representative examples of such silicone elastomers are taught in U.S. Pat. No. 5,880,210 and U.S. Pat. No. 5,760,116. To improve the compatibility of silicone elastomers with various personal care ingredients, alkyls, polyether, amines or other organofunctional groups may be grafted onto the silicone elastomer backbone. Representative examples of such organofunctional silicone elastomers are taught in U.S. Pat. No. 5,811,487, U.S. Pat. No. 5,880,210, U.S. Pat. No. 6,200,581, U.S. Pat. No. 5,236,986, U.S. Pat. No. 6,331,604, U.S. Pat. No. 6,262,170, U.S. Pat. No. 6,531,540, U.S. Pat. No. 6,365,670, WO 2004/104013 and WO 2004/103326.

Other organomodified silicones which may be used in accordance with the invention may be silicone quaternary ammonium compounds or monoquaternary ammonium functional derivatives of alkanolamino polydimethylsiloxanes, such as disclosed in U.S. Pat. No. 5,026,489. The derivatives are exemplified by (R⁹₃SiO)₂Si R⁹—(CHR¹⁰)_{c}NR¹⁰_{d}R¹¹_{3-d} wherein R⁹ is an alkyl group, R¹⁰ is H, alkyl, or aryl, R¹¹ is (CHR¹⁰)OH, c is 1 to 10, and d is 1 to 3.

Other organomodified silicones which may be used in accordance with the invention may be saccharide-siloxane copolymer having a saccharide component and an organosiloxane component and linked by a linking group, such as described in WO 2006/127883, EP 1885331 and US 2008/0199417. The saccharide-siloxane copolymer has the following formula:

R¹²ₑR¹³₍₃₋ₑ₎SiO—[(SiR¹²R¹³O)ₘ—(SiR¹²O)ₙ]ᵥ—SiR¹³₍₃₋ₑ₎R¹²ₑ

wherein each R¹³ may be the same or different and comprises hydrogen, C₁-C₁₂ alkyl, an organic radical, or R³—W, W comprises an epoxy, cycloepoxy, primary or secondary amino, ethylenediamine, carboxy, halogen, vinyl, allyl, anhydride, or mercapto functionality, m and n are integers from 0 to 10,000 and may be the same or different, each e is independently 0, 1, 2, or 3, v is an integer such that the copolymer has a molecular weight less than 1 million, R¹² has the formula Z-(G¹)_{f}-(G²)_{g}, and there is at least one R¹² per copolymer, wherein G¹ is a saccharide component comprising 5 to 12 carbons, f+g is 1-10, f or g can be 0, G² is a saccharide component comprising 5 to 12 carbons additionally substituted with organic or organosilicon radicals, Z is the linking group and is independently selected from the group consisting of:

R¹⁵—NHC(O)—R¹⁶—;

R¹⁵—NHC(O)O—R¹⁶—;

R¹⁵—NH—C(O)—NH—R¹⁶—;

R¹⁵—C(O)—O—R¹⁶—;

R¹⁵—O—R¹⁶—;

R¹⁵—CH(OH)—CH₂—O—R¹⁶—;

R¹⁵—S—R¹⁶—;

R¹⁵—CH(OH)—CH₂—NH—R¹⁶—; and

R¹⁵—N(R¹)—R¹⁶—, and

R¹⁵ and R¹⁶ are divalent spacer groups comprising (R¹⁷)ᵣ(R¹⁸)ₛ(R¹⁹)ₜ, where at least one of r, s and t must be 1, and R¹⁷ and R¹⁹ are either C₁-C₁₂ alkyl or ((C₁-C₁₂)O)ₖ where k is any integer 1-50 and each (C₁-C₁₂)O may be the same or different, R¹⁸ is —N(R²⁰)—, where R²⁰ is H or C₁-C₁₂ alkyl, or is Z—X where Z is previously defined or R¹⁵.

X is a carboxylic acid, phosphate, sulfate, sulfonate or quaternary ammonium radical, and at least one of R¹⁵ and R¹⁶ must be present in the linking group and may be the same or different, and wherein the saccharide-siloxane copolymer is a reaction product of a functionalized organosiloxane polymer and at least one hydroxy-functional saccharide such that the organosiloxane component is covalently linked via the linking group, Z, to the saccharide component.

The saccharide-siloxane copolymer which may be used in accordance with the invention may be ionically-modified saccharide siloxane copolymers, such as described in WO 2006/127924.

The organopolysiloxane may contain any number or combination of M, D, T, or Q units, but has at least one substituent that is a sulfonate group having the general formula:

R²¹-G-(CO)—Ph—SO₃⁻Y⁺

where;

R²¹ is a divalent organic group bonded to the organopolysiloxane; Y is hydrogen, an alkali metal, or a quaternary ammonium; G is an oxygen atom, NH, or an NR²² group where R²² is a monovalent organic group, and Ph is a phenyl cycle.

The sulfonate group substituent is bonded to the organopolysiloxane via a Si—C bond by the R²¹ moiety. The sulfonate group substituent may be present in the organopolysiloxane via linkage to any organosiloxy unit, that is, it may be present on any M, D, or T siloxy unit. The sulfonate functional organopolysiloxane may also contain any number of additional M, D, T, or Q siloxy units of the general formula (R²²₃SiO_{0.5}), (R²²₂SiO), (R²²SiO_{1.5}), or (SiO₂), where R²² is a monovalent organic group, providing that the organopolysiloxane has at least one siloxy unit with the sulfonate functional group present.

The monovalent organic groups represented by R²² in the organopolysiloxanes may have from 1 to 20 carbon atoms, alternatively 1 to 10 carbon atoms, and are exemplified by, but not limited to alkyl groups such as methyl, ethyl, propyl, butyl, pentyl, hexyl, heptyl, octyl, undecyl, and octadecyl; cycloalkyl such as cyclohexyl; aryl such as phenyl, tolyl, xylyl, benzyl, and 2-phenylethyl; amine functional organic groups such as aminopropyl and aminoethylaminoisobutyl; a polyalkylene oxide (polyether) such as polyoxyethylene, polyoxypropylene, polyoxybutylene, or mixtures thereof, and halogenated hydrocarbon groups such as 3,3,3-trifluoropropyl, 3-chloropropyl, and dichlorophenyl. Typically, at least 50 percent, alternatively at least 80%, of the organic groups in the organopolysiloxane may be methyl (denoted as Me).

The R²¹ group in the sulfonate group substituent may be any divalent organic group, but typically is a divalent hydrocarbon group containing 2 to 6 carbon atoms. Divalent hydrocarbons are represented by an ethylene, propylene, butylene, pentylene, or hexylene group. Alternatively, R²¹ is a propylene group, —CH₂CH₂CH₂— or an isobutylene group, —CH₂CH(CH₃)CH₂—.

G in the general formula for the sulfonate substituent group above is an oxygen atom, NH, or an NR²² group where R²² is a monovalent organic group. When G is an N R²² group, R²² may be any of the monovalent organic groups described above. Typically, G is the NH chemical unit forming an amide group in the sulfonate substituent formula above.

Where the silicones used in the home and personal care compositions according to the invention are insoluble in the compositions, they may be used in the form of preformed emulsified particles.

Emulsions which are preferred for use in accordance with the invention are those made via in situ polymerization of the siloxane polymer as taught for example in EP 0874017, where there is described a method of making a silicone-in-water emulsion by mixing at least one polysiloxane having reactive end groups, at least one organosilicon material that reacts with said polysiloxane by a chain extension reaction and a metal containing catalyst for said chain extension reaction to form a composition (I), and then mixing composition (I) with at least one surfactant and water and emulsifying the mixture. The obtained mean particle size is indicated as being greater than 0.3 μm, preferably in the range of about 0.3 to 100 μm, and the viscosity of the silicone is stated as being greater than 10⁵ mm²/s, preferably in the range of 10⁶ to 10⁸ mm²/s at 25° C. Additional information for such emulsions useful in cosmetic skin care applications are described in WO 2002/062311 where the dispersed silicone has a viscosity of at least 100 million mm²/s, and a particle size of at most 1 μm.

The particularly preferred silicones used in compositions according to the invention are selected from non-volatile silicones chosen from the family of polyalkylsiloxanes containing trimethylsilyl end groups, polyalkylsiloxanes containing dimethylsilanol end groups, such as dimethiconol, polysiloxanes containing amine groups, such as amodimethicones or trimethylsilylamodimethicones, polysiloxanes containing polyethylenoxy and/or polypropylenoxy groups, hydrocarbyl functional organopolysiloxane and mixtures of two or more of these. Also useful are emulsions of any of these and in situ polymerized emulsions.

Home and personal care compositions according to the invention include skin care compositions, hair care compositions and home care compositions. Skin care compositions include shower gels, soaps, hydrogels, creams, lotions and balms and may be in the form of water-in-oil emulsion, oil-in-water emulsion, water-in-silicone emulsion, silicone-in-water emulsion or multiple emulsions such as water-in-oil-in-water or oil-in-water-in-oil. Such skin care compositions may contain additional ingredients as appropriate, for example colorants, UV absorbers, antiperspirants, fragrances, antimicrobial or antibacterial or antifungal agents, pigments, preservatives, sunscreens, vegetable or botanical extracts, vitamins and/or moisturizers, surface active materials such as surfactants or detergents, as well as a cosmetically acceptable medium, such as water, emulsifiers and thickeners. Hair care compositions include shampoos, rinse off conditioners, leave-in conditioners, gels, pomades and cuticle coats. Where hair care compositions are under the form of emulsions, they may be water-in-oil emulsion, oil-in-water emulsion, water-in-silicone emulsion, silicone-in-water emulsion. Such hair care compositions may contain additional ingredients as appropriate, for example colorants, dyes, UV absorbers, preservatives, vegetable extracts, fatty alcohols, vitamins, fragrance, anti-dandruff agents, colour care additives, pearlising agents, suspending agents, surface active materials such as surfactants or detergents, thickeners and a cosmetically acceptable medium, such as water. Home care compositions include liquid detergents, solid detergents, fabric softeners and hard surface cleaners. Particular home care compositions of interest are fabric care compositions. Such home care compositions may contain additional ingredients as appropriate, for example colorants, preservatives, fragrance, antifoam compounds, surfactants, antibacterial or antifungal agents, abrasives, enzymes, optical brighteners, colour care additives, dyes transfer inhibitors, dye sequestrants, colour fixatives, anti redeposition agents, bleaching agents, surface active materials such as surfactants or detergents, thickeners and a medium such as water.

The amount of cationic hydrophobically modified galactomannan ether present in compositions according to the invention will be determined by the particular benefit to be obtained, for example, conditioning of the hair, skin or fabric. The particular level appropriate in different compositions according to the present invention is influenced by the particular composition into which it is formulated and by the choice of silicone used. Fine tuning of the relevant amounts of cationic hydrophobically modified galactomannan ether and silicone is preferably done by trial and error. Nevertheless the general level of cationic hydrophobically modified galactomannan ether in the compositions according to the invention may vary from 0.01 to 10% by weight, preferably 0.05 to 2%, most preferably 0.1 to 1%. And the general level of silicone may vary from 0.01 to 10%, preferably 0.5 to 5%, most preferably 1 to 3%. The weight ratio of cationic hydrophobically modified galactomannan ether to silicone is preferably in the range of 5:1 to 1:20, preferably 1:1 to 1:12, most preferably 1:4 to 1:10.

The home and personal care compositions according to the invention are prepared by mixing the cationic hydrophobically modified galactomannan ether in the aqueous phase and the silicone in the appropriate oil or aqueous phase, then mixing respective phases together. Alternatively the galactomannan ether and silicone may be mixed together and formed into an emulsion, before additional ingredients may be added. The silicone and cationic hydrophobically modified galactomannan ether may be present together in the aqueous phase. Mixing devices are those generally used by the man skilled in the art to prepare home and personal care compositions and include mixing vessels with paddles, stirrers, homogenisers, scrapers and other equipment which is known to the person skilled in the art. The process may be performed at temperatures ranging from 15 to 90° C., preferably at room temperature (25° C.). The solubilisation of the cationic hydrophobically modified galactomannan ether in water may be performed at pH ranging from 3 to 9, preferably at pH 4 to 7.

The application of the home and personal care compositions according to the invention generally generate conditioning benefit of the substrate, skin, hair, surface or fabric. Benefits obtained from using the hair care compositions according to the invention include one or more of the following benefits: hair conditioning, softness, detangling ease, silicone deposition. Benefits obtained from using the skin care compositions according to the invention include one or more of the following benefits: skin softness, suppleness. Benefits obtained from using the home care compositions according to the invention include one or more of the following benefits: fabric softening, ease of ironing, colour care, anti-wrinkle, silicone deposition.

The invention also comprises a method of treating a hair, skin or fabric substrate by applying to it a composition according to the first aspect of the invention.

The invention is illustrated by the following examples, in which parts and percentages are by weight and viscosities are measured at 25° C., unless otherwise indicated.

### EXAMPLE 1

100 g of commercial guar flour, having Brookfield viscosity RVT at 20° C., 20 rpm and 1% by weight in water of 7000 mPa·s, is fed into a suitable steel reactor able to resist pressures up to 10 atm, evacuated and filled three times with nitrogen, and then mixed carefully with 0.3 g of sodium tetraborate decahydrate and 8.8 g of sodium hydroxide in 47.5 g of a hydro-alcoholic solvent containing 53% by weight of isopropyl alcohol. After 30 minutes of stirring at 40° C., 0.5 g of hexadecylglycidylether (83% active content) dispersed in 12.5 g of hot isopropyl alcohol are added. The reactor is stirred until the mixture is substantially homogeneous and then heated to 70° C. for 1.5 hours. After this period, the reaction mixture is allowed to cool to 40° C. and the pressure released, and 25.6 g of 3-chloro-2-hydroxypropyltrimethylammoniumchloride are added with 35.1 g of water and the reactor is heated up to 60° C. This temperature is kept for 1.5 hours under constant stirring. At the end of reaction, the alcohol is distilled, the mixture is allowed to cool to 40° C. and a wet product obtained. Then 100 g of it are purified adding by 830 g of ambient temperature water, mixing for 10 minutes and filtering off under vacuum. After drying in a hot air stream at 85-90° C. for 20 minutes, the product is ground and sieved through 100 mesh. The resulting polymer is a hydrophobically modified cationic guar having hexadecyl and cationic degree of substitution of 6.0×10⁻⁴ and 0.15 respectively, and Brookfield viscosity RVT at 20° C., 20 rpm and 1% by weight in water of 5000 mPa·s.

### EXAMPLE 2

A cationic guar with a C₂₂ hydrophobic chain is obtained with the same procedure described in Example 1, starting from commercial guar flour, having Brookfield viscosity RVT at 20° C., 20 rpm and 1% by weight in water of 7000 mPa·s, except that instead of 0.5 g hexadecylglycidylether (83% active content), 0.5 g of C₂₂ alkyl glycidylether (85% active content) was used. The resulting polymer is a hydrophobically modified cationic guar having C₂₂ and cationic degree of substitution of 5.8×10⁻⁴ and 0.14 respectively, and Brookfield viscosity RVT at 20° C., 20 rpm and 1% by weight in water of 5150 mPa·s.

### EXAMPLE 3

Cationic C₁₆ hydrophobically modified hydroxypropylguar is obtained from the commercial guar flour used in Examples 1 and 2, having Brookfield viscosity RVT at 20° C., 20 rpm and 1% by weight in water of 7000 mPa·s. This was obtained by adding to a suitable steel reactor able to resist pressures up to 10 atm, after three vacuum-nitrogen cycles to remove oxygen, 100 g of guar flour are added. Then 0.4 g of sodium tetraborate decahydrate, dissolved in 25 g of sodium hydroxide 30% by weight aqueous solution, were added and carefully mixed. This mixture was stirred for 30 minutes at 40° C. Then 0.3 g of hexadecylglycidylether (83% active content), dispersed in 18.8 g of hot isopropyl alcohol, were added and mixed until homogeneity. The reactor mass was heated to 70° C. and held at this temperature for 1.5 hours. After this period, the reaction mixture was allowed to cool to 40° C. and the pressure released. Then 23.1 g of 3-chloro-2-hydroxypropyltrimethylammoniumchloride were added with 25 g of water and the reactor was stirred for 15 minutes. Then 12.5 g of propylene oxide were added and the mixture heated to 70° C. over a period of 30 minutes, then 22.5 g of propylene oxide were added in 30 minutes. This temperature was kept for 1.5 hours under constant stirring. Then the pressure was released and the alcohol removed, the mixture allowed to cool to 40° C. and the wet product obtained. 100 g of it were purified adding 830 g of ambient temperature water, mixed for 10 minutes and filtered off under vacuum. After drying in a hot air stream at 85-90° C. for 20 minutes, the product was ground and sieved through 100 mesh. The resulting polymer was a hydrophobically modified cationic hydroxypropylguar having C₁₆ and cationic degree of substitution of 6.6×10⁻⁵ and 0.12 respectively, hydroxypropyl molar substitution of 0.42 and Brookfield viscosity RVT at 20° C., 20 rpm and 1% by weight in water of 2150 mPa·s.

### EXAMPLES 4 TO 6 AND COMPARATIVE EXAMPLES 1 TO 4

Shampoo compositions based on cationic hydrophobically modified galactomannan ethers made in Examples 1 to 3 and a non-ionic emulsion of divinyldimethicone/dimethicone copolymer were prepared following details given in Table 1 for Examples 4 and 5 and Comparative example 1 and in Table 2 for Example 6 and Comparative examples 2 to 4.

A shampoo wash was carried out by applying about 4 g of each composition to 10 g of slightly bleached hair previously made wet (5 tresses of 2 g). The shampoo was worked into a lather and then rinsed out thoroughly with water. The initiation of foaming was very easy and the foam was airy. Panellists were asked to disentangle tresses while time is measured. The average recorded times and the standard deviations are given under the corresponding compositions. Results indicate the various combinations with the cationic hydrophobically modified galactomannan ethers according to the invention are substantially equivalent to the Comparative examples. In Table 1, Examples 4 and 5 are equivalent to each other and equivalent to Comparative example 1. In Table 2, Example 6 and Comparative examples 2 and 3 are equivalent, but significantly better than Comparative example 4. Wet hair treated with the composition according to the invention disentangles easily and is supple. Dried hair is smooth and has body and manageability.

Process to produce shampoo compositions of Table 1 to 5:

1) Disperse the cationic hydrophobically modified galactomannan ether in water
2) Add the surfactants under moderate mixing
3) Add the preservative mixture
4) Add silicone when appropriate
5) Adjust pH if necessary

TABLE 1

| | | | Comparative |
|---|---|---|---|
| Ingredient (weight %) | Example 4 | Example 5 | example 1 |
| Sodium laureth sulfate | 33.0 | 33.0 | 33.0 |
| (Texapon NSO by Cognis | | | |
| Corp.) | | | |
| Water | 53.0 | 53.0 | 53.0 |
| Non ionic emulsion of | 3.3 | 3.3 | 3.3 |
| Divinyldimethicone/dimethicone | | | |
| copolymer | | | |
| Cocamidopropyl betaine | 7.0 | 7.0 | 7.0 |
| (Amonyl 380 BA by | | | |
| Seppic) | | | |
| Cocamide DEA (Comperlan | 3.0 | 3.0 | 3.0 |
| KD by Cognis Corp.) | | | |
| Preservative mixture | 0.2 | 0.2 | 0.2 |
| Citric acid - pH to 5.5-6 | q.s. | q.s. | q.s. |
| Polymer from Example 1 | 0.5 | | |
| Polymer from Example 2 | | 0.5 | |
| 2-hydroxy-3- | | | 0.5 |
| trimethylammonium propyl | | | |
| ether chloride | | | |
| Average detangling time (s) | 29.81 | 30.44 | 31.65 |
| Standard deviation (s) | 10.23 | 9.39 | 11.19 |

TABLE 2

| | | Compar- | Compa- | Compar- |
|---|---|---|---|---|
| | | ative | ative | ative |
| Ingredient (weight %) | Example 6 | example 2 | example 3 | example 4 |
| Sodium laureth sulfate | 33.0 | 33.0 | 33.0 | 33.0 |
| (Texapon NSO by | | | | |
| Cognis Corp.) | | | | |
| Water | 53.0 | 53.0 | 53.0 | 53.0 |
| Non ionic emulsion of | 3.3 | 3.3 | 3.3 | 3.3 |
| Divinyldimethicone/ | | | | |
| dimethicone copolymer | | | | |
| Cocamidopropyl | 7.0 | 7.0 | 7.0 | 7.0 |
| betaine (Amonyl 380 | | | | |
| BA by Seppic) | | | | |
| Cocamide DEA | 3.0 | 3.0 | 3.0 | 3.0 |
| (Comperlan KD by | | | | |
| Cognis Corp.) | | | | |
| Preservative mixture | 0.2 | 0.2 | 0.2 | 0.2 |
| Citric acid - pH to 5.5-6 | q.s. | q.s. | q.s. | q.s. |
| Polymer from Example 3 | 0.5 | | | |
| Hydroxypropyl guar | | 0.5 | | |
| hydroxypropyltrimonium | | | | |
| chloride | | | | |
| Guar | | | 0.5 | |
| hydroxypropyltrimonium | | | | |
| chloride | | | | |
| Polyquaternium-7 | | | | 0.5 |
| Average detangling | 24.58 | 28.13 | 27.68 | 45.01 |
| time (s) | | | | |
| Standard deviation (s) | 10.52 | 5.65 | 14.43 | 12.14 |

### EXAMPLES 7 TO 13

Shampoo compositions based on cationic hydrophobically modified galactomannan ethers described in Examples 1 to 3 and on various silicone polymers and emulsion were prepared following details given in Table 3 to Table 5. Each Table contains shampoo compositions based on a combination of 1 specific cationic hydrophobically modified galactomannan with 2 or 3 different types of silicones.

A shampoo wash was carried out by applying about 4 g of each composition to 10 g of slightly bleached hair previously made wet (5 tresses of 2 g). The shampoo was worked into a lather and was then rinsed out thoroughly with water. The initiation of foaming was very easy and the foam was airy. Panellists were asked to disentangle tresses while time was measured. The average recorded time and the standard deviations are given under the respective formulations. Results generally indicate the various combinations of cationic hydrophobically modified galactomannan ethers and silicone may be optimized upon need via a trial and error process. Generally, wet hair treated with the compositions disentangles easily and is supple, more or less depending on the specific combination of silicone and cationic hydrophobically modified galactomannan ether. Dried hair is generally smooth, smoothness intensity being dependent on the cationic hydrophobically modified galactomannan ether and silicone combination.

TABLE 3

| Ingredient (weight %) | Example 7 | Example 8 |
|---|---|---|
| Sodium laureth sulfate | 33.0 | 33.0 |
| (Texapon NSO by Cognis | | |
| Corp.) | | |
| Water | To 100 | To 100 |
| Cocamidopropyl betaine | 7.0 | 7.0 |
| (Amonyl 380 BA by | | |
| Seppic) | | |
| Cocamide DEA | 3.0 | 3.0 |
| (Comperlan KD by | | |
| Cognis Corp.) | | |
| Preservative mixture | 0.2 | 0.2 |
| Citric acid - pH to 5.5-6 | q.s. | q.s. |
| Cyclohexasiloxane | 2 | |
| Alkyl methyl siloxane | | 2 |
| Polymer from Example 1 | 0.5 | 0.5 |
| Average detangling time | 11.63 | 16.48 |
| (s) | | |
| Standard deviation (s) | 5.91 | 4.37 |

TABLE 4

| Ingredient (weight %) | Example 9 | Example 10 |
|---|---|---|
| Sodium laureth sulfate (Texapon NSO | 33.0 | 33.0 |
| by Cognis Corp.) | | |
| Water | to 100 | to 100 |
| Cocamidopropyl betaine (Amonyl 380 | 7.0 | 7.0 |
| BA by Seppic) | | |
| Cocamide DEA (Comperlan KD by | 3.0 | 3.0 |
| Cognis Corp.) | | |
| Preservative mixture | 0.2 | 0.2 |
| Citric acid - pH to 5.5-6 | q.s. | q.s. |
| Amodimethicone | 2 | |
| Dimethicone and Trisiloxane | | 2 |
| Polymer from Example 2 | 0.5 | 0.5 |
| Average detangling time (s) | 6.81 | 16.22 |
| Standard deviation | 1.70 | 6.47 |

TABLE 5

| | Example | Example | Example |
|---|---|---|---|
| Ingredient (weight %) | 11 | 12 | 13 |
| Sodium laureth sulfate (Texapon | 33.0 | 33.0 | 33.0 |
| NSO by Cognis Corp.) | | | |
| Water | to 100 | to 100 | to 100 |
| Cocamidopropyl betaine (Amonyl | 7.0 | 7.0 | 7.0 |
| 380 BA by Seppic) | | | |
| Cocamide DEA (Comperlan KD by | 3.0 | 3.0 | 3.0 |
| Cognis Corp.) | | | |
| Preservative mixture | 0.2 | 0.2 | 0.2 |
| Citric acid - pH to 5.5-6 | q.s. | q.s. | q.s. |
| Dimethicone 100cs | | | 2 |
| Cyclopentasiloxane and Dimethiconol | | 2 | |
| Bis-Hydroxyethoxypropyl | 2 | | |
| Dimethicone | | | |
| Polymer from Example 3 | 0.5 | 0.5 | 0.5 |
| Average detangling time (s) | 6.69 | 13.41 | 19.73 |
| Standard deviation | 2.18 | 4.28 | 7.49 |

### EXAMPLE 14 AND COMPARATIVE EXAMPLE 5

Example 14 fabric care composition was prepared by adding a non ionic emulsion of polydimethylsiloxane in combination with the cationic hydrophobically modified galactomannan ether of Example 3 at respectively 2% and 0.1% in a test liquid detergent. Comparative example 5 was prepared by adding only 2% of the non ionic emulsion of polydimethylsiloxane in the test liquid detergent. 16 panellists evaluated the softness of terry towels treated with the liquid detergents of Example 14 and Comparative example 5. The cationic hydrophobically modified galactomannan ether of Example 3 significantly increased the deposition of the non-ionic emulsion of polydimethylsiloxane positively impacting the softness effect, rating it with an average of 6.63/10.

### EXAMPLES 15 AND 16 AND COMPARATIVE EXAMPLE 6

Shower gel compositions were prepared as described in Table 6, using different combinations of cationic hydrophobically modified galactomannan ethers and silicones, at 0.2% and 2% active level respectively. 4 Panellists were asked to compare various parameters between each of the Example 15 and 16 and Comparative example 6. Example 15 had creamier foam than Comparative example 6. Example 16 provided a smoother after feel on skin than Comparative example 6. These examples show that combinations can be optimized with regard to the cationic hydrophobically modified galactomannan ether and silicone combination depending on the desired benefit either on the foam quality or on the skin feel.

TABLE 6

| | | | Compar- |
|---|---|---|---|
| | Example | Exam- | ative |
| Ingredient (weight %) | 15 | ple 16 | example 6 |
| Water | 47.3 | 48.6 | 50.8 |
| Polymer from Example 3 | 0.2 | — | — |
| Polymer from Example 1 | — | 0.2 | — |
| Citric Acid | qs | qs | qs |
| Sodium laureth sulfate (Empicol | 30 | 30 | 30 |
| ESB 3 by Albright & Wilson | | | |
| UK Limited) | | | |
| Decyl glucoside (Oramix NS 10 by | 5 | 5 | 5 |
| Seppic) | | | |
| Cocamidopropyl Betaine (Amonyl 380 | 10 | 10 | 10 |
| BA by Seppic) | | | |
| Laureth-4 (Brij 30 by Croda Uniqema) | 2 | 2 | 2 |
| Polyacrylamide and C13-14 isoparaffin | 2 | 2 | 2 |
| and laureth-7 (Sepigel 305 by Seppic) | | | |
| Preservative | 0.2 | 0.2 | 0.2 |
| Non ionic emulsion of | 3.3 | — | — |
| Divinyldimethicone/dimethicone | | | |
| copolymer | | | |
| Bis(C13-15 Alkoxy) PG | — | 2 | — |
| Amodimethicone | | | |

Process to produce shower gel compositions of Table 6:

1. Disperse the cationic hydrophobically modified galactomannan ether in water
2. Add the surfactants, the thickener and the preservative under moderate mixing
3. Add silicone when appropriate
4. Adjust pH if necessary
As of here—new examples!

### EXAMPLES 17 TO 19 AND COMPARATIVE EXAMPLE 7

Hair treatment compositions were prepared as described in Table 7, using different combinations of cationic hydrophobically modified galactomannan ethers and silicones.

TABLE 7

| | | | | Compar- |
|---|---|---|---|---|
| | Example | Example | Example | ative |
| Ingredient (weight %) | 17 | 18 | 19 | example 7 |

Phase A

| | | | | |
|---|---|---|---|---|
| Cetearyl Alcohol | 5 | 5 | 5 | 5 |

Phase B

| | | | | |
|---|---|---|---|---|
| Water | 88.5 | 88.5 | 89.5 | 88.5 |
| Polymer from example 1 | 0.5 | — | — | — |
| Polymer from example 2 | — | 0.5 | — | — |
| Polymer from example 3 | — | — | 0.5 | — |
| Guar hydroxypropyl | — | — | — | 0.5 |
| trimonium chloride | | | | |
| Glycerin | 1.5 | 1.5 | 1.5 | 1.5 |

Phase C

| | | | | |
|---|---|---|---|---|
| Bis-Isobutyl PEG/PPG- | — | 4 | — | 4 |
| 20/35/Amodimethicone | | | | |
| Copolymer | | | | |
| (and) Cetyl | | | | |
| Ethylhexanoate (and) | | | | |
| Polysorbate 80 | | | | |
| Silicone quaternium-16 | 4 | — | — | — |
| (and) Undeceth-11 (and) | | | | |
| Butyloctanol (and) | | | | |
| Undeceth-5 | | | | |
| Bis-Hydroxy/Methoxy | — | — | 3 | — |
| Amodimethicone | | | | |

Phase D

| | | | | |
|---|---|---|---|---|
| Preservative mixture | 0.5 | 0.5 | 0.5 | 0.5 |

Process to produce hair treatment compositions of Table 7:

1. Melt phase A at 80° C.
2. Mix phase B ingredients together and heat to 80° C.
3. Add phase A to phase B with high shear mixing

### 4. Cool to 45° C.

5. Add phase C with mixing
6. Add phase D and mix until homogeneous

### EXAMPLES 20 TO 22 AND COMPARATIVE EXAMPLE 8

Leave in compositions were prepared as described in Table 8, using different combinations of cationic hydrophobically modified galactomannan ethers and silicones.

TABLE 8

| | | | | Compar- |
|---|---|---|---|---|
| | Example | Example | Example | ative |
| Ingredient (weight %) | 20 | 21 | 22 | example 8 |

Phase A

| | | | | |
|---|---|---|---|---|
| Water | 85.8 | 80.66 | 85.8 | 77.8 |
| Polymer from example 1 | 0.5 | — | — | — |
| Polymer from example 2 | — | 0.5 | — | — |
| Polymer from example 3 | — | — | 0.5 | — |
| Guar hydroxypropyl | — | — | — | 0.5 |
| trimonium chloride | | | | |

Phase B

| | | | | |
|---|---|---|---|---|
| Tetrasodium EDTA | 0.2 | 0.2 | 0.2 | 0.2 |
| Glycerin | 11 | 11 | 11 | 11 |
| Amodimethicone (and) | — | — | — | 10 |
| C11-15 Pareth-7 (and) | | | | |
| Laureth-9 | | | | |
| (and) glycerin (and) | | | | |
| Trideceth-12 | | | | |
| Bis(C13-15 Alkoxy) | 2 | — | — | — |
| PG-Amodimethicone | | | | |
| Silicone Quaternium- | — | 7.14 | — | — |
| 16/Glycidoxy | | | | |
| Dimethicone | | | | |
| crosspolymer | | | | |
| (and) Trideceth-12 | | | | |
| PEG/PPG-15/15 | — | — | 2 | — |
| Dimethicone | | | | |
| Preservative mixture | 0.5 | 0.5 | 0.5 | 0.5 |

Process to produce leave in compositions of Table 8:

1. Prepare phase A by dispersing polymer in water
2. Add ingredients of phase B in order

### EXAMPLES 23 TO 26 AND COMPARATIVE EXAMPLES 9 AND 10

Rinse off compositions were prepared as described in Table 9, using different combinations of cationic hydrophobically modified galactomannan ethers and silicones. Compositions have similar viscosities as measured using a Brookfield apparatus. The compositions were applied to hair and panellists were asked to disentangle tresses while time was measured. The average recorded time and the standard deviations are given under the respective formulations. Results indicate the various combinations of cationic hydrophobically modified galactomannan ethers and silicone are very effective hair conditioners. Wet hair treated with the compositions disentangles easily and is supple. Dried hair is generally smooth, smoothness intensity being dependent on the cationic hydrophobically modified galactomannan ether and silicone combination.

TABLE 9

| | | | | | Com- | Com- |
|---|---|---|---|---|---|---|
| | | | | | par- | par- |
| | | | | | ative | ative |
| | Ex- | Ex- | Ex- | Ex- | ex- | ex- |
| Ingredient | ample | ample | ample | ample | ample | ample |
| (weight %) | 23 | 24 | 25 | 26 | 9 | 10 |

Phase A

| | | | | | | |
|---|---|---|---|---|---|---|
| Hydroxyethyl | 1.0 | 1.0 | 1.0 | 1.0 | 1.5 | 1.0 |
| cellulose | | | | | | |
| (Natrosol | | | | | | |
| 250HHR by | | | | | | |
| Hercules) | | | | | | |
| Polymer from | 0.7 | — | — | — | — | — |
| example 1 | | | | | | |
| Polymer from | — | 0.7 | 0.7 | — | — | — |
| example 2 | | | | | | |
| Polymer from | — | — | — | 0.7 | — | — |
| example 3 | | | | | | |
| Guar | — | — | — | — | — | 0.7 |
| hydroxy- | | | | | | |
| propyl | | | | | | |
| trimonium | | | | | | |
| chloride | | | | | | |
| Water | 50.0 | 50.0 | 50.0 | 50.0 | 50.0 | 50.0 |

Phase B

| | | | | | | |
|---|---|---|---|---|---|---|
| Cetyl alcohol | 1.0 | 1.0 | 1.0 | 1.0 | 1.0 | 1.0 |
| PEG-100 | 1.0 | 1.0 | 1.0 | 1.0 | 1.0 | 1.0 |
| stearate & | | | | | | |
| glyceryl | | | | | | |
| stearate | | | | | | |
| Bis (C13-15 | 2.0 | — | — | — | 2.0 | 2.0 |
| Alkoxy) PG | | | | | | |
| amodimeth- | | | | | | |
| icone | | | | | | |

Phase C

| | | | | | | |
|---|---|---|---|---|---|---|
| Silicone | — | 6.67 | — | — | — | — |
| quaternium-16 | | | | | | |
| (and) | | | | | | |
| Undeceth-11 | | | | | | |
| (and) | | | | | | |
| Butyloctanol | | | | | | |
| (and) | | | | | | |
| Undeceth-5 | | | | | | |
| Bis-Isobutyl | — | — | 10 | — | — | — |
| PEG/PPG- | | | | | | |
| 20/35/ | | | | | | |
| Amodimeth- | | | | | | |
| icone | | | | | | |
| Copolymer | | | | | | |
| (and) Cetyl | | | | | | |
| Ethyl- | | | | | | |
| hexanoate | | | | | | |
| (and) | | | | | | |
| Polysorbate 80 | | | | | | |
| Amodimeth- | — | — | — | 4 | — | — |
| icone (and) | | | | | | |
| Cetrimonium | | | | | | |
| chloride (and) | | | | | | |
| Trideceth-12 | | | | | | |
| Water | 44.3 | 39.6 | 36.3 | 42.3 | 44.5 | 44.3 |

Phase D

| | | | | | | |
|---|---|---|---|---|---|---|
| Citric acid | q.s. | q.s. | q.s. | q.s. | q.s. | q.s. |
| (20%) | | | | | | |
| Viscosity after | 14.100 | 11.500 | 10.700 | 10.100 | 11.800 | 15.200 |
| 24 hours | | | | | | |
| (Brookfield) | | | | | | |
| in cPs | | | | | | |
| Average | 6.02 | 4.19 | 5.21 | 4.69 | 7.02 | 6.23 |
| detangling | | | | | | |
| time (s) | | | | | | |
| Standard | 3.62 | 1.64 | 2.48 | 2.74 | 2.48 | 2.20 |
| deviation | | | | | | |

Process to produce rinse off compositions of Table 9:

1. Prepare phase A
2. Combine phase B ingredients and heat to 80-85° C.
3. Heat phase C to 80-85° C. in the final beaker
4. Add phase B to phase C with rapid stirring
5. Mix for 5-10 minutes
6. Add phase A (at room temperature) slowly to the hot emulsion
7. Continue mixing until the batch cools to 40-45° C.
8. Compensate the water loss when room temperature is reached
9. Adjust pH to 4 with phase D

### EXAMPLES 27 TO 29 AND COMPARATIVE EXAMPLE 11

Deodorant compositions were prepared as described in Table 10, using different combinations of cationic hydrophobically modified galactomannan ethers and silicones.

TABLE 10

| | Example | Example | Exam- | Comparative |
|---|---|---|---|---|
| Ingredient (weight %) | 27 | 28 | ple 29 | example 11 |

Phase A

| | | | | |
|---|---|---|---|---|
| Water | 94.66 | 94.66 | 94.66 | 94.66 |
| Polymer from example 1 | 1.00 | — | — | — |
| Polymer from example 2 | — | 1.00 | — | — |
| Polymer from example 3 | — | — | 1.00 | — |
| Guar hydroxypropyl | — | — | — | 1.00 |
| trimonium chloride | | | | |

Phase B

| | | | | |
|---|---|---|---|---|
| Triclosan | 0.30 | 0.30 | 0.30 | 0.30 |
| Glycolic acid at 70% | 0.04 | 0.04 | 0.04 | 0.04 |
| Glycerol | 2.00 | 2.00 | 2.00 | 2.00 |
| Bis-PEG-18 Methyl | — | 2.00 | — | 2.00 |
| Ether Dimethyl Silane | | | | |
| Cyclopentasiloxane and | 2.00 | — | — | — |
| Dimethicone | | | | |
| Bis- | — | — | 2.00 | — |
| Hydroxyethoxypropyl | | | | |
| Dimethicone | | | | |

Process to produce deodorant compositions of Table 10:

1. Prepare phase A
2. Add phase B ingredients in order to phase A

### EXAMPLES 30 TO 32 AND COMPARATIVE EXAMPLE 12

Oil-in-water lotion compositions were prepared as described in Table 11, using different combinations of cationic hydrophobically modified galactomannan ethers and silicones. Example 32 is more slippery than Comparative example 12, but both oil-in-water lotions are easy to spread, have low tackiness and low greasiness.

TABLE 11

| | Example | Exam- | Exam- | Comparative |
|---|---|---|---|---|
| Ingredient (weight %) | 30 | ple 31 | ple 32 | example 12 |

Phase A

| | | | | |
|---|---|---|---|---|
| Cetyl Alcohol | 5.0 | 5.0 | 5.0 | 5.0 |
| Glycol Distearate | 1.0 | 1.0 | 1.0 | 1.0 |
| Mineral oil | 7.0 | 7.0 | 7.0 | 7.0 |
| Cocamide DEA | 0.5 | 0.5 | 0.5 | 0.5 |
| (Comperlan KD by | | | | |
| Cognis Corp.) | | | | |
| Stearalkonium Chloride | 3.0 | 3.0 | 3.0 | 3.0 |
| Dimethicone | — | — | 3.0 | 3.0 |
| C30-45 Alkyldimethylsilyl | 3.0 | — | — | — |
| Polypropylsilsesquioxane | | | | |

Phase B

| | | | | |
|---|---|---|---|---|
| Water | 78.2 | 78.2 | 78.2 | 78.2 |
| Polymer from example 1 | 1.0 | — | — | — |
| Polymer from example 2 | — | 1.0 | — | — |
| Polymer from example 3 | — | — | 1.0 | — |
| Guar hydroxypropyl | — | — | — | 1.0 |
| trimonium chloride | | | | |

Phase C

| | | | | |
|---|---|---|---|---|
| Divinyldimethicone/ | — | 3.0 | — | — |
| Dimethicone Copolymer | | | | |
| (and) C12-13 Pareth-23 | | | | |
| (and) C12-13 Pareth-3 | | | | |
| Cyclopentasiloxane (and) | 1.0 | 1.0 | 1.0 | 1.0 |
| Cyclohexasiloxane | | | | |
| Preservative mixture | 0.3 | 0.3 | 0.3 | 0.3 |

Process to produce oil-in-water lotion compositions of Table 11:

1. Heat phase A ingredients to 70-75° C.
2. Mix phase B ingredients together and heat to 70° C.
3. Slowly add phase A to phase B
4. Cool to approximately 40° C. with stirring
5. Add phase C ingredients in order with mixing
6. Continue mixing with same speed, until homogeneous

### EXAMPLES 33 TO 35 AND COMPARATIVE EXAMPLE 13

Shaving gel compositions were prepared as described in Table 12, using different combinations of cationic hydrophobically modified galactomannan ethers and silicones.

TABLE 12

| | Example | Example | Exam- | Comparative |
|---|---|---|---|---|
| Ingredient (weight %) | 33 | 34 | ple 35 | example 13 |

Phase A

| | | | | |
|---|---|---|---|---|
| Cyclopentasiloxane | 7.0 | 7.0 | 7.0 | 7.0 |
| (and) PEG/PPG-18/18 | | | | |
| Dimethicone | | | | |
| Trisiloxane (and) | 8.0 | 8.0 | 8.0 | 8.0 |
| Dimethicone | | | | |
| Cyclopentasiloxane | 8.0 | 8.0 | 8.0 | 8.0 |
| (and) Dimethicone | | | | |
| Crosspolymer | | | | |

Phase B

| | | | | |
|---|---|---|---|---|
| Water | 76.0 | 76.0 | 76.0 | 76.0 |
| Polymer from example 1 | 1.0 | — | — | — |
| Polymer from example 2 | — | 1.0 | — | — |
| Polymer from example 3 | — | — | 1.0 | — |
| Guar hydroxypropyl | — | — | — | 1.0 |
| trimonium chloride | | | | |
| Viscosity after 24 hours | 9.400 | 14.000 | 7.300 | 7.550 |
| (Brookfield) in cPs | | | | |

Process to produce shaving gel compositions of Table 12:

1. Prepare phase A
2. Prepare phase B
3. Add phase B to phase A

### EXAMPLES 36 TO 38 AND COMPARATIVE EXAMPLE 14

Shaving gel compositions were prepared as described in Table 13, using different combinations of cationic hydrophobically modified galactomannan ethers and silicones.

TABLE 13

| | Example | Example | Exam- | Comparative |
|---|---|---|---|---|
| Ingredient (weight %) | 36 | 37 | ple 38 | example 14 |

Phase A

| | | | | |
|---|---|---|---|---|
| Dimethicone (and) | 7.0 | 7.0 | 7.0 | 7.0 |
| PEG/PPG 18/18 | | | | |
| Dimethicone | | | | |
| Dimethicone | 8.0 | 8.0 | 8.0 | 8.0 |
| Isodecyl Neopentanoate | 8.0 | 8.0 | 8.0 | 8.0 |
| (and) Dimethicone/Bis- | | | | |
| Isobutyl PPG-20 | | | | |
| Crosspolymer | | | | |

Phase B

| | | | | |
|---|---|---|---|---|
| Water | 76.0 | 76.0 | 76.0 | 76.0 |
| Polymer from example 1 | 1.0 | — | — | — |
| Polymer from example 2 | — | 1.0 | — | — |
| Polymer from example 3 | — | — | 1.0 | — |
| Guar hydroxypropyl | — | — | — | 1.0 |
| trimonium chloride | | | | |
| Viscosity after 24 hours | 8.350 | 8.800 | 8.400 | 7.800 |
| (Brookfield) in cPs | | | | |

Process to produce shaving gel compositions of Table 13:

1. Prepare phase A
2. Prepare phase B
3. Add phase B to phase A

### EXAMPLES 39 TO 44

Water-in-silicone compositions were prepared as described in Table 14, using different combinations of cationic hydrophobically modified galactomannan ethers and silicones. The sensory profile of Example 41 describes a cream that is easy to spread, that has low tack, medium absorbency, medium gloss, slight greasiness, and a smooth, slippery feel. The sensory profile of Example 44 describes a cream that is easy to spread, that has low tack, rapid absorbency, low shine, low greasiness, medium smoothness and medium slipperiness.

TABLE 14

| | Ex- | Ex- | Ex- | Ex- | Ex- | Ex- |
|---|---|---|---|---|---|---|
| | ample | ample | ample | ample | ample | ample |
| Ingredient (weight %) | 39 | 40 | 41 | 42 | 43 | 44 |

Phase A

| | | | | | | |
|---|---|---|---|---|---|---|
| PEG-10 Dimethicone | 2.0 | 2.0 | 2.0 | 2.0 | 2.0 | 2.0 |
| Dimethicone | 11.0 | 11.0 | 11.0 | 11.0 | 11.0 | 11.0 |
| Isododecane | — | — | — | — | — | — |
| Pentaerythrityl | 6.0 | 3.0 | 3.0 | 3.0 | 3.0 | 3.0 |
| tetraethylhexanoate | | | | | | |
| Caprylyl Methicone | 11.0 | 11.0 | 11.0 | 11.0 | 6.0 | 9.0 |
| Phenyl Trimethicone (and) | — | 2.0 | — | — | — | — |
| Dimethiconol | | | | | | |
| C13-16 Isoparaffin (and) | — | — | 3.0 | — | — | — |
| Dimethicone | | | | | | |
| Isododecane (and) | — | — | — | — | — | 5.0 |
| Dimethicone Crosspolymer | | | | | | |
| C30-45 Alkyldimethylsilyl | — | — | — | 3.0 | — | — |
| Polypropylsilsesquioxane | | | | | | |

Phase B

| | | | | | | |
|---|---|---|---|---|---|---|
| Water | 64.5 | 65.5 | 64.5 | 64.5 | 69.5 | 64.5 |
| Polymer from example 1 | — | — | — | — | — | 0.5 |
| Polymer from example 2 | 0.5 | 0.5 | 0.5 | — | — | — |
| Polymer from example 3 | — | — | — | 0.5 | 0.5 | — |
| Divinyldimethicone/ | — | — | — | — | 3.0 | — |
| Dimethicone Copolymer | | | | | | |
| (and) C12-13 Pareth-23 | | | | | | |
| (and) C12-13 Pareth-3 | | | | | | |
| Glycerin | 5.0 | 5.0 | 5.0 | 5.0 | 5.0 | 5.0 |
| Viscosity after 24 hours | 120 | 150 | 170 | 2.900 | 110 | 380 |
| (Brookfield) in cPs | | | | | | |

Process to produce water-in-silicone compositions of Table 14:

### 1. Prepare Phase A

2. Prepare phase B
3. Add phase B to phase A with high shear

### EXAMPLES 45 TO 47 AND COMPARATIVE EXAMPLES 15 AND 16

Emulsions were prepared as described in Table 15, using combinations of cationic hydrophobically modified galactomannan ethers and dimethicone, to potentially be used as raw material in other cosmetic compositions to facilitate process by adding 1 component containing the conditioning polymers to the final formulations.

TABLE 15

| Ingredient | Comparative | Comparative | Example | Example | Example |
|---|---|---|---|---|---|
| (weight %) | example 15 | example 16 | 45 | 46 | 47 |
| Water | 30.96 | 30.39 | 30.20 | 30.42 | 29.87 |
| Polymer from | — | — | 1.07 | — | — |
| example 1 | | | | | |
| Polymer from | — | — | — | 1 | — |
| example 2 | | | | | |
| Polymer from | — | — | — | — | 1.05 |
| example 3 | | | | | |
| Guar | — | 0.54 | — | — | — |
| hydroxypropyl | | | | | |
| trimonium | | | | | |
| chloride | | | | | |
| Cocamidopropyl | 3.48 | 3.69 | 3.54 | 3.55 | 3.57 |
| betaine | | | | | |
| C12-15 pareth-3 | 0.55 | 0.55 | 0.58 | 0.58 | 0.6 |
| Dimethicone | 64.13 | 63.83 | 63.74 | 63.55 | 63.99 |
| Phenoxyethanol | 0.88 | 0.98 | 0.87 | 0.89 | 0.91 |
| Viscosity | 590 | 34600 | 145000 | 155000 | 175000 |
| (Brookfield) cPs | | | | | |
| Particle size | 21.3 | 15.0 | 113.6 | 24.7 | 22.7 |
| (Malvern) dv 0.5 μm | | | | | |

Process to produce emulsions of Table 15:

1. Mix for a few minutes Part I of water (±17.5%) and Guar
2. Add Part I of cocamidopropyl betaine (±1.6%) and mix (hand)
3. Add C12-15 pareth-3 and mix (hand)
4. Add dimethicone and mix 3 time with the speedmixer
5. Add Part II of water (±11.2%) and mix 2 times with the speedmixer
6. Add Part III of water (±5.4%) and mix 2 times with the speedmixer
7. Add Part IV of water (±5.4%) and mix 2 times with the speedmixer
8. Add Part II of cocamidopropyl betaine (rest %) and the phenoxyethanol
9. Mix 1 time with the speedmixer

1. A home and personal care composition comprising a silicone and a cationic hydrophobically modified galactomannan ether have a cationic degree of substitution (DS_{cat}) from 0.01 to 0.5 and a hydrophobic degree of substitution (DS_{H}) from 0.00001 to less than 0.001.

2. A home and personal care composition according to claim 1, wherein the cationic substitution of the galactomannan ether is obtained from the reaction of one of the hydroxyl group of the galactomannan with tertiary amino or quaternary ammonium alkylating agents.

3. A home and personal care composition according to claim 1, wherein the hydrophobic substitution of the galactomannan ether is obtained by the introduction of hydrophobic substituents selected from the group consisting of linear or branched alkyl and/or alkenyl substituents of from 12 to 32 carbon atoms either alone or mixed together.

4. A home and personal care composition according to claim 1, wherein the galactomannan ether has a cationic substituent which is 2-hydroxy-3-(trimethylammonium)propyl chloride and a hydrophobic substituent which comprises a linear chain alkyl containing between 14 and 24 carbon atoms.

5. A home and personal care composition according to claim 1, wherein the silicone is selected from silicone oils, silicone waxes, silicone resins, silicone elastomers and silicone gums.

6. A home and personal care composition according to claim 1, wherein the silicone is present in the form of an emulsion.

7. A home and personal care composition according to claim 1, wherein the silicone is selected from non-volatile silicones chosen from the family of polyalkylsiloxanes containing trimethylsilyl end groups, polyalkylsiloxanes containing dimethylsilanol end groups, polysiloxanes containing amine groups, polysiloxanes containing polyethylenoxy and/or polypropylenoxy groups, hydrocarbyl functional organopolysiloxane and mixtures of two or more of these

8. A personal care composition according to claim 1, which is a skin care composition.

9. A personal care composition according to claim 8, which is a shower gel, soap, hydrogel, cream, lotion or balm.

10. A personal care composition according to claim 1, which is a hair care composition.

11. A personal care composition according to claim 10, which is a shampoo, rinse off conditioner, leave-in conditioner, gel, pomade or cuticle coat.

12. A home care composition according to claim 1, which is a fabric care composition, a liquid detergent, a solid detergent, a fabric softener or a hard surface cleaner.

13. A home and personal care composition according to claim 1, wherein the cationic hydrophobically modified galactomannan ether is present in an amount of from 0.01 to 10% by weight and the silicone is present in an amount of from 0.01 to 10% by weight, and wherein the weight ratio of cationic hydrophobically modified galactomannan ether to silicone is in the range of 5:1 to 1:20.

14. A method of treating a hair, skin or fabric substrate by applying to it a composition comprising a silicone and a cationic hydrophobically modified galactomannan ether have a cationic degree of substitution (DS_{cat}) from 0.01 to 0.5 and a hydrophobic degree of substitution (DS_{H}) from 0.00001 to less than 0.001.
